(19) United States
(12) Patent Application Publication
(10) Pub. No.: US 2005/0065458 A1
(43) Pub. Date: Mar. 24, 2005

(54) Splint combined use cast absence for bone fracture fixing

(76) Inventor: Kyung-Jin Kim, Yesan oup Yesan-gun (KR)

Correspondence Address: FAY, SHARPE, FAGAN, MINNICH & MCKEE, LLP, 1100 SUPERIOR AVENUE, SEVENTH FLOOR, CLEVELAND, OH, 44114, US

(21) Appl. No.: 10/500,439

(22) PCT Filed: Dec. 27, 2002
(86) PCT No.: PCT/KR02/02449

(30) Foreign Application Priority Data
Dec. 28, 2001 (KR) 10-2001-0087054

(51) Int. Cl. A61F 5/00

(52) U.S. Cl. 602/5

(57) ABSTRACT

Disclosed is a combined splint and cast for immobilizing the injured body part due to fracture, ligament rupture, dislocation or the like. In particular, the present invention relates to a combined splint and cast for immobilizing fractured bones, in which a splint and a cast used for a predetermined period of time until a swelling in the injured body part subsides are injec-tion-molded to be combined into one. Therefore, the present invention can solve inconvenience of working with the conventional plaster cast member by using a foot fixing member, a connecting member and a knee fixing member of the combined splint and cast individually or cooperatively according to the use, for example, a short leg splint, a Patella Tendon Bearing (PTB) splint or a long leg splint, minimize the sequelae, such as joint contracture by making possible early joint movement, and correct an inaccurate reduction at its initial stage at any time and malunion by making intermediate inspections.

### TECHNICAL FIELD

The present invention relates to a combined splint and cast for immobilizing the injured body part due to fracture, ligament rupture, dislocation or the like. In particular, the present invention relates to a combined splint and cast for immobilizing fractured bones, in which a splint and a cast used for a predetermined period of time until a swelling in the injured body part subsides are injection-molded to be combined into one. Therefore, the present invention can solve inconvenience of working with the conventional plaster cast member by using a foot fixing member, a connecting member and a knee fixing member of the combined splint and cast individually or cooperatively according to the use, for example, a short leg splint, a Patella Tendon Bearing(PFB) splint or a long leg splint, minimize the sequelae, such as joint contracture by making possible early joint movement, and frequently check an inaccurate reduction at its initial stage and malunion by making intermediate inspections.

### BACKGROUND ART

In general, when bones are fractured, cracked, or ligaments are lengthened or ruptured, an orthopedic splint needs to be applied for the purpose of tightly supporting and immobilizing the injured joints and muscles partially or entirely.

Material used for the splint needs to be flexible enough to be easily molded to conform to the curved shape of the injured body part, requires a proper amount of cure time, be easy to handle, and has mechanical strength high enough to maintain the structural strength after the cure. One of widely used methods using the qualified material is to wind a plaster bandage on the injured body part. Since such plaster bandage has high flexibility before solidification such that it can be easily molded to conform to the shape of the injured body part, requires a proper amount of cure time and work time, and has high mechanical strength, it has been widely used for a long time.

The plaster bandage, however, has a problem in that it has to be wound on the injured body part in sufficient thickness, thereby taking lots of time to be wound. To avoid the problem, presently, there has been developed a splint in which wet-cure-type one component polyurethane resin is coated on a plaster bandage, glass fiber or synthetic fabric, and the resultant coating is repeatedly wound into multi layers, thereby reducing the time taken to be used. When the splint constructed as above is applied to the curved body part, such as elbow, knee, heel or the like, it has to be bent to conform to the angle of the injured body part. Inevitably, it gets crumpled on the curved part. Further, since it is made in a long rectangular form, it fails to closely wrap the injured body part in accordance with the thickness of the curved shape, e.g., the wrist, arm, ankle and calf. In this case, the splint gets loose from the injured body part and fails to tightly support the injured body part, thereby badly affecting the fractured part.

Furthermore, the method using the plaster bandage has problems in that since the work process is complicated and the plaster bandage is wound over the injured body part, mold or infectious bacteria may grow. In addition, the process of cutting the cast by using a saw to remove the cast raises the dust.

In recent years, to obviate those disadvantages Utility Model Publication No. 1995-13343 entitled a cast member for fracture patients and Utility Model Publication No. 2001-0016563 entitled a splint structure disclose a method of molding thermoplastic resin into a thin sheet and adhering the inner surface of a sponge to the resin. In those disclosures, a splint is put into an oven and gets softened at the temperature of 90° C or so. The splint is put on the injured body part and closely attached to the injured body part along the curved surface by being pressed with hands. Since the soften splint made of thermoplastic resin gets cool during this forming process, it cannot be closely attached to the injured body part at once completely. For the reason, the splint has to be put into the oven more than one time to be softened and cooling time of about 20 minutes is required after the forming process is finished so as for the splint to become cold completely. Thus, the splint causes inconvenience in application and takes lots of time, resulting in poor practicality.

A short leg splint is used to immobilize the foot. A PTB splint is used to immobilize the region from the foot to a portion below the knee. A long leg splint is used to immobilize the region from the foot to the middle portion of the femur. Before a cast is applied, the splints are used for a predetermined period of time to lessen the swelling in the injured body part. In the conventional art, since the splint and the cast are separately applied, inconveniently materials for the splint and the cast are separately prepared and applied.

Moreover, when the cast is applied on the injured body part, early joint movement is difficult to be done during the cast period, and inaccurate reduction and abnormal fixation cannot be checked through intermediate inspections.

### DISCLOSURE OF INVENTION

Accordingly, the present invention is directed to a combined splint and cast for fixing fractured bones that substantially obviates one or more problems due to limitations and disadvantages of the related art.

An object of the present invention is to provide a combined splint and cast for fixing fractured bones which can fast and easily fix the fractured bones by injection-molding a foot fixing member, a connecting member, and a knee fixing member of various sizes according to body measurements and using the respective members individually or cooperatively according to the use, for example, a short leg splint, a PTB splint or a long leg splint, and reduce the time consumed to apply the cast and decrease the cost for the cast application by combining a splint and a cast into one and omitting the intermediate process of applying the plaster cast.

Another object of the present invention is to provide a combined splint and cast for fixing fractured bones which can obtain a precise immobilization effect by eliminating difference in cast application skill between surgical operators and assistants, and prevent side effects, such as contamination caused by mold or infectious bacteria on the cast portion by providing a plurality of ventilation holes on the respective members so as to allow smooth air circulation in the injured body part, differently from the conventional method in which the cast entirely encloses the injured body part.

A further object of the present invention is to provide a combined splint and cast for fixing fractured bones which can minimize joint contracture and reduce duration of treatment by making early rehabilitation and early joint movement possible during the cast period, achieve a precise union by performing intermediate checks on initial inaccurate reduction or abnormal fixation, and make a patient feel comfortable during movement by using the respective members made of light synthetic resin.

A still further object of the present invention is to provide a combined splint and cast for fixing fractured bones which can perform a radioactive examination with the cast on by using radial-ray-permeable material for the cast, and perform a surgical operation while performing medical treatment to patients ho has an open fracture or open reduction with open wounds.

To achieve these objects and other advantages, there is provided a combined splint and cast for immobilizing the injured body part due to fracture, ligament rupture, dislocation or the like, the combined splint and cast comprising: a foot fixing member including the upper foot fixing member and the lower foot fixing member which form a pair for immobilizing the region from the sole (metatarsal bones) to the portion above the ankle (below the soleus muscle); a connecting member including the upper connecting member and the lower connecting member which form a pair for immobilizing the region from the ankle to the portion below the knee; and a knee fixing member for immobilizing the region from the knee to the middle portion of the femur, wherein the foot fixing member, the connecting member and the knee fixing member can be used individually or cooperatively by being coupled to each other by means of bolts.

It is to be understood that both the foregoing general description and the following detailed description of the present invention are exemplary and explanatory and are intended to provide further explanation of the invention as claimed.

### BRIEF DESCRIPTION OF THE DRAWINGS

Further objects and advantages of the invention can be more fully understood from the following detailed description taken in conjunction with the accompanying drawings in which:

FIG. 1 is a front view illustrating the human skeletal system;

FIG. 2 is an exploded perspective view illustrating the whole structure of the present invention according to the present invention;

FIG. 3 is a perspective view illustrating the lower foot fixing member in use according to the present invention;

FIG. 4 is a perspective view illustrating a foot fixing member in use according to the present invention;

FIG. 5 is a perspective view illustrating the lower connecting member in use according to the present invention;

FIG. 6 is a perspective view illustrating a connecting member in use according to the present invention; and

FIG. 7 is a perspective view illustrating a knee fixing member in use according to the present invention.

### BEST MODE FOR CARRYING OUT THE INVENTION

The present invention will now be described in detail in connection with preferred embodiments with reference to the accompanying drawings. For reference, like reference characters designate corresponding parts throughout several views.

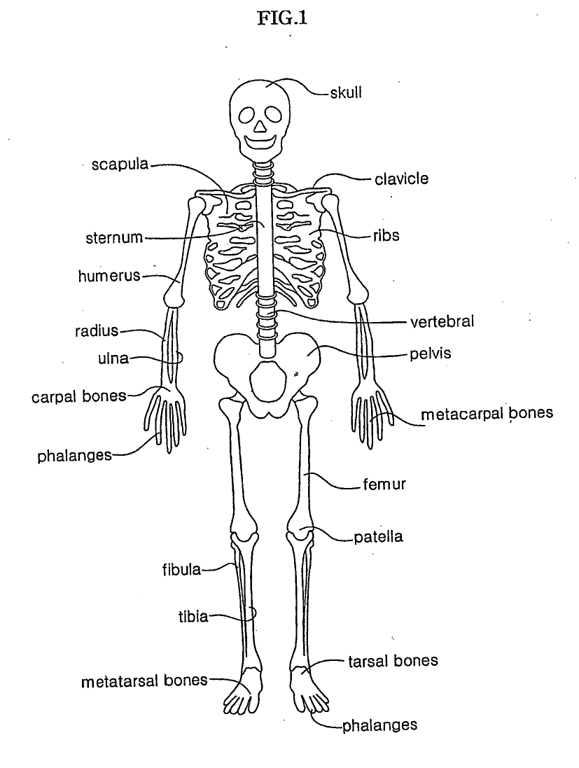

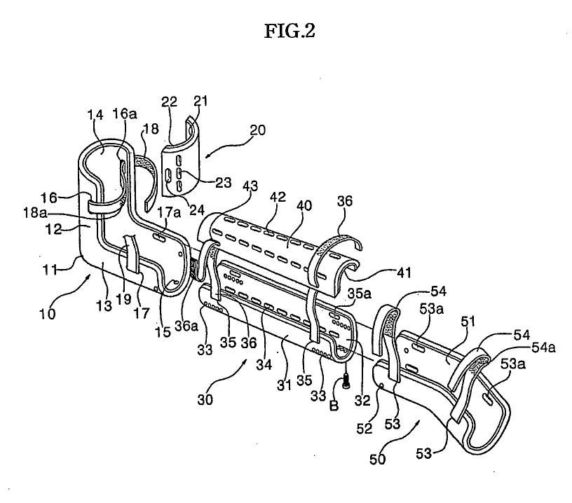

FIG. 1 is a front view illustrating the human skeletal system, and FIG. 2 is an exploded perspective view of a combined splint and cast according to the present invention.

Referring to FIGS. 1 and 2, the combined splint and cast is used when fracture occurs in the leg. Since leg bones bear the body weight, if one person has a fractured leg, he or she is difficult to walk and needs crutches or a walking stick. In this case, surgical operation is carried out after selecting the methods for fixing and reducing the fractured bones. The combined splint and cast used t each portion includes a foot fixing member 10 having the upper and lower foot fixing members 20 and 11 for immobilizing the region from the metatarsophalangeal (MP) joint to the portion above the heel (below the soleus muscle), a connecting member 30 connected to the foot fixing member 10 at one end thereof for immobilizing the region from the ankle to the portion below the knee, and a knee fixing member 50 connected to the connecting member 30 for immobilizing the region from the knee to the middle portion of the femur, wherein the foot fixing member 10, the connecting member 30 and the knee fixing member 50 can be used individually or cooperatively.

The foot fixing member 10 is mainly as a short leg splint in case of fracture in tarsal bones and metatarsal bones.

The bones are most frequently fractured when a heavy object is dropped down on the foot. As shown in FIG. 1, the tarsal bones mean seven irregular bones between the ankle and the heel, and the metatarsal bones mean five long bones situated at one end of the tarsal bones, namely, between the toes and the. When the tarsal bones and the metatarsal bones are injured, a splint is primarily used, in which the splint is fixed after selecting the foot fixing member of a size corresponding to the specific body part (foot) of a patient.

The foot fixing member 10 includes the upper foot fixing member 20 and the lower foot fixing member 11 which form a pair for wrapping the region from the sole (metatarsal bones) to a portion above the ankle (below the soleus muscle). The respective members are injection molded according to the foot size based on the research result on standardized body dimensions.

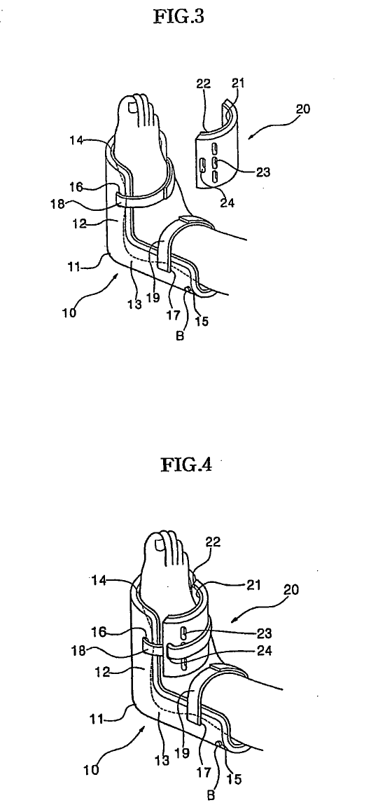

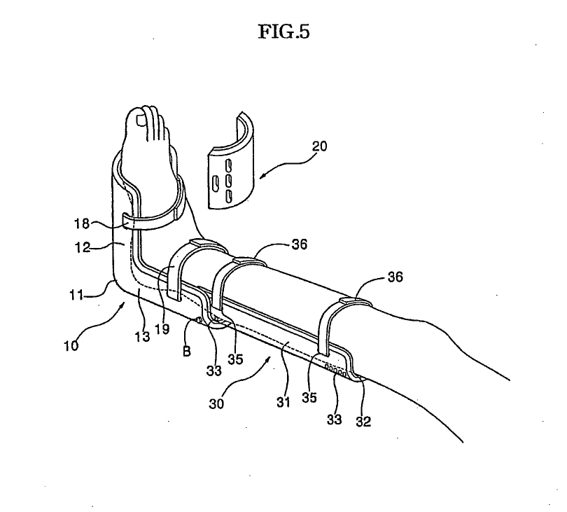

The lower foot fixing member 11, as shown in FIGS. 3 and 4, includes a vertical wall 12, a horizontal wall 13 and a buffering material 14. The lower foot fixing member 11 is formed in such a manner that the horizontal wall 13 in which the heel and the ankle are seated is bent from the vertical wall 12 in which the sole is seated and the horizontal wall 13, and the buffering material 14 made of sponge is arranged in the inner face of the lower foot fixing member 11 for softly wrapping the foot.

A plurality of bolt holes 15 are formed at the front portion on the inner surface of the horizontal wall 13. One end of the connecting member 30 is inserted into the lower foot fixing member such that the connecting member 30 and the lower foot fixing member are coupled to each other with bolts B inserted through the bolt holes 15. Holes 16, 16a are formed at opposite lateral sides of the vertical wall 12 and holes 17 and 17a are formed at opposite lateral sides of the horizontal wall 13, that is, at both side surfaces which are contacted by the foot when the foot is placed. One end of a fastener tape 18 is fixed to one hole formed at one side of the vertical wall 12 and the other end of the fastener tape is fixed to holes 24 formed in the upper foot fixing member 20 disposed to cover the top of the foot (metatarsal bones) so that the coupled condition between the upper foot fixing member 20 and the lower foot fixing member 11 is adjusted.

The connecting member 30 connected to the horizontal wall 13 of the lower foot fixing member 11 can be connected in several ways. For instance, the connecting member can be connected using an instantaneous adhesive.

Referring to FIG. 4, the upper foot fixing member 20 is used in cooperation with the lower foot fixing member 11 when the cast is worn on the foot. The upper foot fixing member 20 has a round part 21 in close contact with the top of the foot (metatarsal bones), a buffering material 22 arranged in the inside thereof for softly wrapping the top of the foot, and a plurality of ventilation holes 23 formed on the inner peripheral surface thereof such that external air is communicated with the foot through the ventilation holes 23, thereby preventing contamination caused by mold or infectious bacteria.

The holes 24 are formed on both lateral surfaces in the middle portion of the upper foot fixing member 20 such that the other end of the fastener tape 18 is inserted into the holes 24 and accordingly couples the upper foot fixing member 20 to the lower foot fixing member 11. To closely couple the upper foot fixing member 20 to the lower foot fixing member 11, the other end of the fastener tape 18 passes through the holes 24 of the upper foot fixing member 20 and is inserted into the hole 16a of the lower foot fixing member 11. Alternatively, two fastener tapes 18 are provided on both sides of the lower foot fixing member 11 and fix the upper and lower fixing members 20 and 11 with the fastener tapes 18. Herein, an adhesive part 18a is formed from the middle portion of the outer surface of the fastener tape 18 to the distal end of the fastener tape 18 such that the distal end of the fastener tape 18 is fastened as illustrated in FIGS. 3 and 4, leading to coupling the upper foot fixing member 20 to lower foot fixing members 11.

The connecting member 30 is mainly used when a PTB splint is required. When there occurs fracture in the tibia shaft, the connecting member 30 is used to immobilize the fractured bones and help to take early movement of the knee joint.

The fracture occurring in the fibula and the tibia shaft is often generated at all ages. It happens when a direct or indirect force is applied to the fibula and the tibia shaft. In many cases, an open fracture occurs due to a direct external force or when fractured pieces of the tibia close to the surface of the skin are protruded through the skin. The case due to the direct external force is most frequently generated by a car accident.

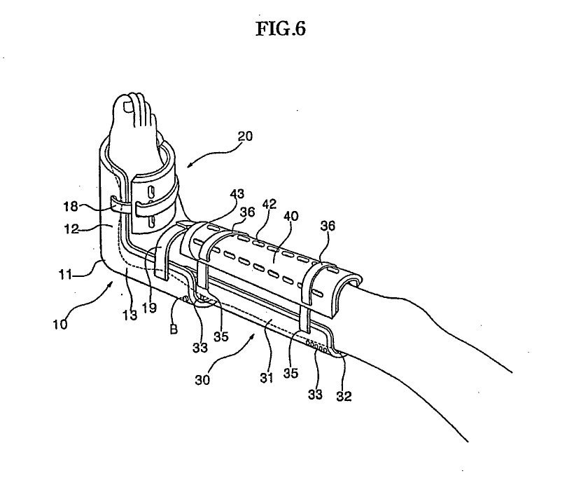

The connecting member 30, as shown in FIGS. 5 and 6, includes the upper connecting member 40 and the lower connecting member 31 which have the same structure and form a pair for wrapping the region from the ankle (under the soleus muscle) to the portion below the knee. The connecting member 30 is shaped in conformity with the human body and injection-molded based on the research result on standardized body dimensions.

The connecting member 30 is formed in such a manner as to entirely enclose the tibia and the fibula from the ankle (tarsal bones) via the calf to a portion below the knee (below the patella). The lower connecting member 31 has a buffering material 32 arranged in the inside thereof for wrapping the region from the ankle to the portion below the knee.

The lower connecting member 31 has a plurality of bolt holes 33 formed at the front and rear portions thereof at equal intervals in a length-adjustable manner. The lower connecting member 30 is inserted into the front portion of the lower foot fixing member 11 such that the upper connecting member 30 is coupled to the lower foot fixing member 11 with bolts B inserted through the bolt holes 33. The lower connecting member 31 has a plurality of ventilation holes 34 in a longitudinal direction formed at opposite lateral sides on the inner peripheral surface thereof which are contacted by the calf when the calf is placed in the lower connecting member 31, such that external air is smoothly circulated, thereby preventing contamination caused by mold or infectious bacteria. The lower connecting member 31 has a plurality of holes 35 at opposite lateral sides at front and rear portions thereof, a fastener tape 36 with one end fixed to one hole 35 and the other end inserted into one of holes 43 formed on the upper connecting member 40 which is coupled to the lower connecting member 31, thereby firmly wrapping the tibia and the fibula.

The upper connecting member 40 has a buffering material 41 for wrapping the tibia and the fibula, and a plurality of ventilation holes 42 formed at opposite lateral sides on the inner peripheral surface thereof in a longitudinal direction such that external air is smoothly circulated, thereby preventing contamination of mold or infectious bacteria due to the conventional plaster bandage.

The upper connecting member 40 has the plurality of holes 43 formed at opposite lateral sides in such a manner as to correspond to the holes 35 of the lower connecting member 31. One end of the fastener tape 36 is fixed to one side of the vertical wall 12, and the other end of the fastener tape 36 passes through the holes 43 and is inserted into a hole 35a of the lower connecting member 31 in such a manner as to wrap the outer peripheral surface of the upper connecting member 36. After that, the distal end of the fastener tape 36 is fastened to an adhesive part 36a formed at the outer surface of the fastener tape 36 such that the upper connecting member 40 and the lower connecting member 31 are firmly coupled to each other.

Herein, the fastener tape 36 is used in the above manner or others. By way of example, four fastener tapes 36 are provided on both sides of the front and rear surfaces of the lower connecting member 31 and individually used to fix the upper connecting member 40 to the lower connecting member 31.

Further, the fastener tape 36 used for the connecting member 30 can be substituted by other fastening means, e.g., a belt, a string or the like.

The knee fixing member 40 is applied when fracture occurs in the tibia and the fibula. The knee fixing member 40 is used by being coupled with one end of the connecting member 30 when a long leg splint for immobilizing the region from the knee to the middle portion of the femur is required.

Fracture in the knee (knee joint) includes fracture in the tibia, fracture in the knee joint, and fracture in the femur. The injury to the tibia usually happens as a traumatic injury. In most cases of the fracture in the knee, a compound compression fracture or a depressed fracture happens.

The fracture in the patella occurs due to a direct external force applied to the knee or a sudden contraction of quadriceps femoris muscles. When the external force is applied, there happens a crack or a compound fracture in many cases.

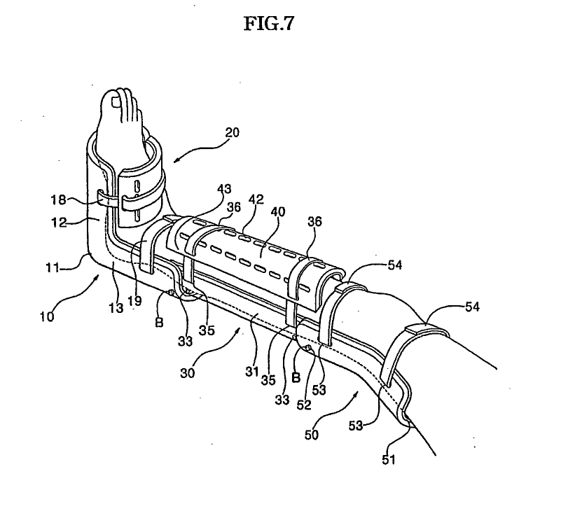

The knee fixing member 50, as shown in FIG. 7, has a buffering material 51 adhered to the inside thereof for immobilizing the region from the knee to the middle portion of the femur, and a plurality of bolt holes 52 formed at the front portion thereof such that the knee fixing member 50 is coupled to the rear surface of the connecting member 30 with bolts B inserted through the bolt holes 52. The knee fixing member 50 has a plurality of holes 53 formed on both opposite lateral sides of the upper part of the front and rear portions thereof such that one end of a fastener tape 54 is fixed to one side of the knee fixing member 50 and the other end of the fastener tape 54 is inserted into holes 53a which correspond to the holes 53 of the knee fixing member to adjust the length of the fastener tape and then the distal end of the fastener tape 54 is fastened to an adhesive surface 54a of the fastener tape 54.

The foot fixing member 10, the connecting member 30 and the knee fixing member 50 are used individually or cooperatively by being fixedly coupled to each other by means of the bolts B.

The steps in which the present invention constructed as above is applied will be explained with reference to FIGS. 2 through 7. First, the respective members injection molded from synthetic resin, that is, the foot fixing member 10, the connecting member 30 and the knee fixing member 50 are used according to the patient's physical conditions on the basis of the research result on standardized body dimensions. Since the present invention combines the splint and the cast, thereby eliminating the necessity of an additional material for splint fixing.

In case of the short leg splint which is required when there is fracture in the tarsal bones or the metatarsal bones, first the foot is put on the buffering material 14 attached to the foot fixing member 10 and then a cotton-bandage (not shown) is disposed between the buffering material 14 and the foot so as to prevent the foot from being moved. After that, the foot fixing member 10 is fastened by means of the fastener tapes 18 and 19 at the metatarsal bones (top of the foot) and the tarsal bones (ankle). When a swelling in the foot subsides to some degree after a certain period of time passes, the upper foot fixing member 20 is positioned on the top of the foot according to the condition of the metatarsal bones to couple the upper foot fixing member 20 to the lower foot fixing member with the fastener tape 18 of the lower foot fixing member 11, thereby conveniently completing the cast application.

In case of the PTB splint, the foot fixing member 10 and the connecting member 30 are used together. First, the foot is positioned in the lower foot fixing member 11 in the same manner as above described. Next, the bolts B are fitted into the bolt holes 15 of the lower foot fixing member 11 according to the patient's physical conditions so as to adjust the length of the lower connecting member 31. As described above, when there is foot movement inside the lower connecting member 31, the cotton bandage is used to prevent any movement of the foot. After that, the fastener tape 35 is wound to fix the lower connecting member 31 to the foot.

When the swelling subsides to some degree after a predetermined period of time passes, the upper foot fixing member 20 and the upper connecting member 40 are coupled to each other according to the patient's condition, thereby conducting the cast application.

In case of the long leg splint, the foot fixing member 10, the connecting member 30 and the knee fixing member 50 are used together. As described above, the foot fixing member 10 is coupled to one end of the connecting member 30 and the knee fixing member 50 is coupled to the other end of the connecting member 30 with the bolts B after adjusting the length of the knee fixing member 50. The fastener tape 54 is wound on the knee and the femur, thereby completing a series of steps for the cast application. It is preferable that the portion of the knee fixing member 50 in which the knee is located is molded to have an inclination of about 5 degrees.

As previously said, the combined splint and cast of the present invention can be injection-molded from materials having a predetermined strength and provided according to size of body part to systematically perform the cast application. In addition, since the cast members are light in weight, the patient doesn't feel inconvenience in walking.

Industrial Applicability

As described above, the present invention relates to a combined splint and cast for immobilizing the injured body part due to fracture, rupture, dislocation, etc. The present invention can reduce the cast application time and cost by injection-molding the foot fixing member, the connecting member and the knee fixing member having various sizes according to the injured condition of the patient on the basis of the research result on standardized body dimensions and making them usable individually or cooperatively.

The present invention can obtain a precise immobilization effect by eliminating difference in cast application skill between surgical operators and assistants, and prevent side effects, such as contamination caused by mold or infectious bacteria observed in the conventional cast portions by providing the plurality of ventilation holes on the respective members and making the respective members separable from each other.

The present invention can decrease the inconvenience that the patient feels during walking by providing the members materialized of light synthetic resin.

The present invention can achieve exact union fast by combining the splint and the cast into one, systematically applying the cast and taking the intermediate checks while the cast is worn on.

The present invention can permit the patient to use a half-splint even after a surgical operation and make possible an early treatment through the intermediate inspections on the injured body part and the early rehabilitation.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A combined splint and cast for immobilizing the injured body part due to fracture, ligament rupture, dislocation or the like, the combined splint and cast comprising:
a foot fixing member including the upper foot fixing member and the lower foot fixing member which form a pair for immobilizing the region from the sole (metatarsal bones) to a portion above the ankle (below the soleus muscle);
a connecting member including the upper connecting member and the lower connecting member which form a pair for immobilizing the region from the ankle to the portion below the knee; and
a knee fixing member for immobilizing the region from the knee to the middle portion of the femur,
wherein the foot fixing member, the connecting member and the knee fixing member can be used individually or cooperatively by being coupled to each other by means of bolts.

2. The combined splint and cast as defined in claim 1, wherein the foot fixing member, the connecting member and the knee fixing member are injection molded.

3. The combined splint and cast as defined in claim 1, wherein the lower foot fixing member of the foot fixing member includes:
a vertical wall in which the sole is seated;
a horizontal wall in which the heel and the ankle are seated for surrounding the foot together with the vertical wall by being bent from the vertical wall;
a plurality of holes formed at the front portion of the horizontal wall such that the connecting member is inserted into the lower foot fixing member up to the front portion of the horizontal wall and the connecting member is coupled to the lower foot fixing member with bolts inserted through the bolt holes;
a plurality of holes formed at opposite lateral sides of the vertical wall and the horizontal wall;
a buffering material arranged in the inner face thereof for wrapping the foot; and
a fastener tape with one end fixed to any one of the holes and the other end fixed to the upper foot fixing member which is formed in such a manner as to cover the top of the foot and be coupled to the vertical wall for adjusting the contact condition between the upper foot fixing member and the lower foot fixing member.

4. The combined splint and cast as defined in claim 1 or 3, wherein the upper foot fixing member of the foot fixing member is used in cooperation with the lower foot fixing member when being used on the foot, and includes:
a round part in a close contact with the top of the foot;
a buffering material arranged in the inner face thereof for softly wrapping the top of the foot;
a plurality of ventilation holes formed on the inner peripheral surface thereof for allowing air circulation; and
a plurality of holes formed on both lateral sides of the middle portion thereof for being fastened by the other end of the fastener tape fixed to the lower fixing member.

5. The combined splint and cast as defined in claim 1, wherein the lower connecting member of the connecting member is adapted to wrap the region from the ankle to a portion below the knee, and includes:
a buffering material arranged in the inner face thereof;
a plurality of bolt holes formed at the front and rear surfaces thereof at equal intervals for allowing length adjustment and fastened to one end of the foot fixing member with bolts;
a plurality of ventilation holes formed at opposite lateral sides on the inner peripheral surface thereof in a longitudinal direction;
a plurality of holes formed at the opposite lateral sides of the front and rear portions thereof; and
a fastener tape with one end fixed to any one of the holes and the other end inserted into a remaining one of the holes for securely wrapping the tibia and the fibula.

6. The combined splint and cast as defined in claim 1 or 5, wherein the upper connecting member of the connecting member includes:
a buffering material arranged in the inner face, thereof for wrapping the upper portion of the tibia and the fibula;
a plurality of ventilation holes formed at the opposite lateral sides on the inner peripheral surface thereof in a longitudinal direction for allowing smooth air circulation; and
a plurality of holes formed at both sides of the front and rear portions of the upper connecting member for being fastened by the other end of the fastener tape which is fixed to the lower connecting member.

7. The combined splint and cast as defined in claim 1, wherein the knee fixing member is positioned beneath the knee, and includes:
a buffering material arranged in the inner face thereof for immobilizing the knee;
a plurality of bolts holes formed at the front portion thereof such that the rear portion of the connecting member is coupled to the knee fixing member with bolts inserted through the bolt holes;
a plurality of holes formed at both sides of the front and rear portions thereof; and
a fastener tape with one end fixed to any one of the holes and the other end coupled with an opposed one of the holes for securely wrapping the knee.
